(19) United States
(12) Patent Application Publication
(10) Pub. No.: US 2025/0235594 A1
(43) Pub. Date: Jul. 24, 2025

(54) TARGETED APHERESIS TO TREAT INFLAMMATION

(71) Applicant: Henry J. Smith, Temecula, CA (US)

(72) Inventor: Henry J. Smith, Temecula, CA (US)

(21) Appl. No.: 18/418,605

(22) Filed: Jan. 22, 2024

(51) Int. Cl. A61M 1/34

(52) U.S. Cl. CPC A61M 1/3496

(57) ABSTRACT

This invention teaches a targeted apheresis device that contains a plurality of different immobilized binding agents that can simultaneously target and bind out multiple proinflammatory factors belonging to three distinct proinflammatory systems: These systems are 1) proinflammatory factors that bind to the cell membrane; 2) the complement system; and 3) proinflammatory cytokines. This invention teaches a unique targeted apheresis device to remove a) C-Reactive Protein (CRP); and b) complement C1q; and c) Tumor Necrosis Factor (TNF); and optionally d) one or more other complement components, and/or one or more other proinflammatory cytokines. In one embodiment of this invention the targeted apheresis device is modified such that it will remove blood leukocytes in addition to removing CRP, C1q and TNF. Removing both cellular and humoral proinflammatory factors will further inhibit the harmful inflammatory process that occurs in many diseases and following tissue injury.

### RELATED APPLICATIONS

This application claims priority to provisional application #U.S. 63/576,224 titled “Targeted Apheresis to treat Inflammation” and filed Jan. 26, 2023. Confirmation no. 1462.

### BACKGROUND TO THE INVENTION

Inflammation is a natural process that occurs when there is a tissue injury caused by trauma, or an infection, or a disease process. In fact inflammation is an integral part of the healing process.

There are however certain situations where the inflammatory process appears to be out-of-control and may play a significant role as a causative agent in certain inflammatory diseases, or as an agent that can exacerbate the disease process for a variety of different diseases. It is also becoming increasingly recognized that chronic inflammation may predispose to ill-health and the development of many common diseases; including for example: obesity; metabolic disease syndrome; inflammatory bowel disease, diabetes, cardiovascular disease, stroke, and possibly Alzheimer's disease. In many of these diseases where there appears to be a dysfunctional overactive inflammatory process a variety of anti-inflammatory medications have been prescribed and found to be of clinical benefit in ameliorating the symptoms of the disease. However certain medications, such as for example corticosteroids, are often accompanied with serious side-effects requiring their discontinuance over time.

This invention teaches an alternative approach to reducing inflammation by removing proinflammatory substances from the blood of the patient using a process of “Targeted Apheresis”.

Targeted apheresis is a process, similar to kidney dialysis, whereby the patient's blood is treated extracorporeally to remove unwanted harmful substances before the treated blood is returned to the patient. Targeted apheresis however differs from kidney dialysis in that the patient's blood is not dialyzed against a dialysis solution, but the blood is instead treated with one or more binding agents that will selectively bind out one or more harmful proinflammatory substances from the blood. The treated blood is then returned to the patient. Reducing the level of harmful substances in the patient's blood will ameliorate the symptoms of the disease and could lead to remission.

The novelty of this invention is its teaching that there are different interlinked systems and multiple factors involved in inflammation; and that the removal of multiple proinflammatory factors belonging to different systems will be more therapeutically effective than the removal of any single proinflammatory factor. This invention teaches a unique targeted apheresis device that contains a plurality of different immobilized binding agents that can simultaneously target and bind out multiple proinflammatory factors belonging to three distinct proinflammatory processes: This invention teaches said targeted apheresis device to specifically remove a) C-Reactive Protein (CRP); b) complement C1q; c) Tumor Necrosis Factor (TNF); and optionally d) one or more other complement components, and/or one or more other proinflammatory cytokines.

The prior art is silent on the use of a single targeted apheresis device to treat pathological inflammation by simultaneously removing CRP, C1q and TNF; and optionally other proinflammatory factors from the blood of the patient.

In one embodiment of this invention the targeted apheresis device incorporates a leukocyte reduction filter to simultaneously remove proinflammatory cells in addition to removing CRP, C1q and TNF; and optionally other proinflammatory factors from the blood of the patient. The prior art is silent on the use of a single targeted apheresis device to treat pathological inflammation by simultaneously removing proinflammatory leukocytes in addition to removing CRP, C1q and TNF; and optionally other proinflammatory factors from the blood of the patient.

### SUMMARY

This invention teaches a targeted apheresis device that contains a plurality of different immobilized binding agents that can simultaneously target and bind out multiple proinflammatory factors belonging to three distinct proinflammatory systems: These systems are 1) proinflammatory factors that bind to the cell membrane; 2) the complement system; and 3) proinflammatory cytokines. This invention teaches a unique targeted apheresis device to remove a) C Reactive Protein (CRP); and b) complement C1q; and c) Tumor Necrosis Factor (TNF); and optionally d) one or more other complement components, and/or one or more other proinflammatory cytokines. In one embodiment of this invention the targeted apheresis device is modified such that it will remove blood leukocytes in addition to removing CRP, C1q and TNF. Removing both cellular and humoral proinflammatory factors will further inhibit the harmful inflammatory process that occurs in many diseases, and/or following tissue injury.

### DESCRIPTION OF THE INVENTION

Inflammation is a natural process that occurs when there is a tissue injury caused by trauma, or an infection, or a disease process. In fact inflammation is an integral part of the healing process.

There are however certain situations where the inflammatory process appears to be out-of-control and may play a significant role as a causative agent after tissue injury, or in certain inflammatory diseases; or as an agent that can exacerbate the disease process for a variety of different diseases; or as a predisposing chronic condition that may lead to ill-health.

Inflammation is a multifactorial process and there are multiple different proinflammatory factors involved in different stages of the inflammatory process. This invention teaches that removing multiple proinflammatory factors involved in the different stages of inflammation will be more effective than removing a single proinflammatory factor. In this invention the term “inflammation” and “inflammatory process” are used in the widest sense to describe all stages of inflammation from inception to resolution; and to encompass all the proinflammatory factors, both cellular and humoral, that are associated with each stage, including the immune response that is involved in the inflammatory process. As noted earlier pathological inflammation may occur subsequent to tissue injury caused by trauma. There are also many diseases where inflammation appears to be a pathogenic factor associated with that disease. Removing multiple proinflammatory factors using targeted apheresis will be generally applicable to treating a variety of inflammatory diseases or conditions where acute or chronic inflammation is involved in the disease process.

In this invention the term “remove” does not mean complete removal of the harmful substance from the blood. It is well known that following apheresis when the treated blood is returned to the patient there is always a residual level of the harmful substance remaining in the blood of the patient no matter how long or efficiently the apheresis treatment is performed. Generally as a rule of thumb there is approximately a 50% reduction in the level of the harmful substance being removed for every 1.5 times the blood volume of the patient is processed during therapeutic apheresis.

The inflammatory process begins when cells are injured. This causes alterations in the cell membrane and a proinflammatory factor called C-Reactive Protein (CRP) binds to the membrane of the injured cell. This activates the complement system to respond, which could lead to the death of the injured cell. The cell injury also stimulates the production of proinflammatory cytokines which exacerbates inflammation, and also stimulates proinflammatory leukocytes to migrate to the site of inflammation and participate in inflammation.

This invention teaches that an imbalance i.e. an excess of one or more of these proinflammatory factors can cause certain types of inflammatory diseases e.g. inflammatory bowel disease; and a “cytokine storm” that can occur in certain viral diseases. An overactive inflammatory process can also potentiate increased cell death where there was tissue injury following a traumatic event such as myocardial infarction, or stroke.

This invention teaches a unique targeted apheresis device to treat a variety of different diseases or chronic conditions by removing multiple proinflammatory factors from a patient's blood. Reducing the level of these harmful factors in the patient's blood will result in amelioration of disease symptoms, and possibly remission of the disease.

### Targeted Apheresis Device

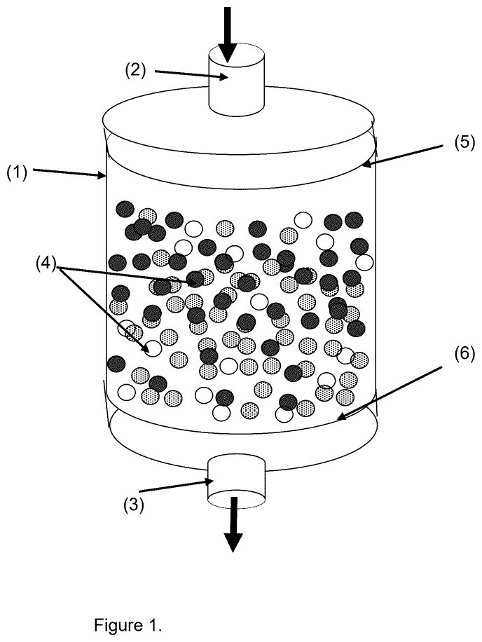

The Targeted Apheresis device disclosed in this invention consists of a rigid container filled with a plurality of different immobilized binding agents; wherein each binding agent will bind out a specific harmful proinflammatory factor. The device illustrated in FIG. 1 is typically in the form of a cylinder or cartridge (1) with an inlet port (2) to allow blood or plasma to enter, and an outlet port (3) to allow the treated blood or plasma to exit and be returned to the patient. Typically the binding agents are attached covalently to micron sized beads (4) that are retained within the device by a microporous membrane at the top (5) and a microporous membrane at the bottom (6) of the device. The pores in the retaining membranes are sufficiently large enough to allow blood or plasma to pass through; but smaller than the diameter of the binding agent coated beads, and will therefore retain them within the device. This invention teaches that there are three different immobilized binding agents used in the Targeted Apheresis Device; a) anti-CRP binding agent coated beads (shown in white); b) anti-C1q binding agent coated beads (shown in black); and c) anti-TNF binding agent coated beads (shown in grey).

The binding agents used in this invention are typically antibodies. However, those of skill in the art will be cognizant that there are other moieties such as aptamers and binding peptides that can also bind to a specific target in a similar manner to that of an antibody. Said aptamers and binding peptides are the functional equivalent of antibodies and can be used as binding agents in the Targeted Apheresis device of this invention. In this invention the term “binding agent” will apply to an antibody, or an aptamer, or a binding peptide.

### Anti-C Reactive Protein (CRP) Binding Agent.

In this invention the anti-CRP binding agent is typically an anti-CRP antibody. However, as noted earlier, an anti-CRP aptamer, or an anti-CRP binding peptide, may also be used as the binding agent.

CRP is an acute phase protein circulating in the blood that increases tremendously when there is an infection or tissue injury. It is widely used as a biomarker to monitor the course of inflammation that accompanies infection, or tissue destruction e.g. myocardial infarct; or immune disorders e.g. rheumatoid arthritis. There is however, growing evidence that CRP is not simply a bystander biomarker associated with many diseases. Instead it now appears that CRP may in fact be a pathogenic agent that participates and directly exacerbates the severity of the disease.

CRP is secreted by hepatocytes in the liver. It circulates in the blood as a pentamer composed of five protomers. The pentamer structure of CRP can disassociate into its individual protomers and each of these is referred to as a “CRP monomer”. Each CRP monomer has two different binding sites. One binding site can bind to the cell membrane of an injured cell to form a CRP/cell membrane complex; and this in turn will cause the C1q component of the complement system to bind to the CRP/cell membrane complex and activate the complement system which would lead to the death of the cell.

The normal level of CRP in serum is typically under 1 mg/L. In response to infection or cell injury the level can rapidly increase within a matter of days to more than 100 mg/L. This invention teaches that reducing the level of CRP using targeted apheresis will reduce the amount of CRP binding to injured cells. This will inhibit the binding of C1q to the CRP/cell membrane complex and thus inhibit the complement system from being activated and killing the injured cells. The amount of cell death is thus reduced and the injured cells are allowed to recover.

### Anti-C1Q Binding Agent.

In this invention the anti-C1q binding agent is typically an anti-C1q antibody. However, as noted earlier, an anti-C1q aptamer, or an anti-C1q binding peptide, may also be used as the binding agent.

The complement system consists of over 40 proteins that act in concert to perform a variety of functions that impact the inflammatory process. When cells are injured the first component of complement to be activated is C1q. It binds to the CRP/cell membrane complex on injured cells and starts the complement cascade process leading to cell death. The normal level of C1q in serum is about 100 mg/L. It can increase markedly when there is tissue injury such as after myocardial infarct or stroke. Removing C1q using targeted apheresis will reduce the amount of C1q available to bind to the CRP/cell membrane complex on injured cells and thereby inhibit the complement process from completion and killing the injured cells.

The complement cascade process involves a number of interdependent steps. Therefore removing one or more other complement components in addition to removing C1q may further inhibit complement activity. In one embodiment of this invention in addition to removing C1q this invention teaches the optional removal of other complement components using targeted apheresis.

### Anti-Tumor Necrosis Factor (TNF) Binding Agent.

In this invention the anti-TNF binding agent is typically an anti-TNF antibody. However, as noted earlier, an anti-TNF aptamer, or an anti-TNF binding peptide, may also be used as the TNF binding agent.

There are a number of proinflammatory cytokines that are known to participate in the inflammatory process. When cells are injured there are a variety of proinflammatory cytokines that are produced including: Tumor Necrosis Factor (TNF), Interleukin 1β (IL-1β), Interleukin 2 (IL-2), Interleukin 6 (IL-6), interleukin 12 (IL-12), interleukin 17 (IL-17), interleukin 18 (IL-18) and interferon-γ (IFN-γ). An abnormally high level of one or more of these proinflammatory cytokines can cause or exacerbate certain diseases that have an inflammatory component; as for example in autoimmune diseases such as rheumatoid arthritis (RA), Systemic lupus erythematosus (SLE), and inflammatory bowel disease (IBD). The occurrence of a “cytokine storm” that can occur in certain viral diseases may also be more prevalent than was formerly thought. In this invention the proinflammatory cytokine-TNF is removed using targeted apheresis.

TNF is a proinflammatory cytokine that is produced in mononuclear phagocytes, lymphocytes, and natural killer cells. Different studies have given markedly different normal range levels of TNF in serum (e.g. from under 3 μg/ml to a mean of 46 μg/ml). However, there is general agreement that where there is inflammation associated with a particular disease there is also a significant increase in the level of circulating TNF. This invention teaches that the removal of TNF using targeted apheresis will prevent it from stimulating other cells from producing more proinflammatory cytokines in a feedback loop resulting in increasing cytotoxicity to injured cells. In order to establish a correlation between the targeted apheresis treatment and inhibition of inflammation it will be necessary to establish a baseline normal range for TNF so that the level of circulating TNF before and after apheresis can be accurately measured and shown to have a beneficial therapeutic effect upon the course of the disease or condition. Because of its multiple effects in promoting inflammation the removal of TNF using targeted apheresis was selected to be an essential step in this invention.

This invention teaches that in addition to TNF there are other proinflammatory cytokines including: Interleukin 1β (IL-1β), Interleukin 2 (IL-2), Interleukin 6 (IL-6), interleukin 12 (IL-12), interleukin 17 (IL-17), interleukin 18 (IL-18), and interferon-γ (IFN-γ) whose removal using targeted apheresis could also inhibit inflammation. In one embodiment of this invention, in addition to the removal of TNF, one or more other proinflammatory cytokines are removed using targeted apheresis.

### Description of Binding Agents

The binding agents used in this invention are antibodies, or aptamers; or binding peptides.

### Antibody as Binding Agent.

In this invention the term “antibody” is used to include polyclonal antibodies, monoclonal antibodies and recombinant antibodies; and also the binding fragments (i.e. Fab) of these antibodies. This invention teaches that either the whole antibody molecule, or the Fab binding fragment of the antibody molecule, is used as the binding agent.

Polyclonal antibodies are prepared by immunizing various species of animals against a particular harmful factor. Typically, the animals used are rabbits, goats, sheep and horses but other animals can also be used. The antisera from immunized animals are treated to isolate and purify the antibodies using established methods including salt-fractionation, gel-filtration and affinity chromatography. These and other methods of purifying antibodies are known to those of skill in the art and are considered to be within the scope of this invention.

Monoclonal antibodies are typically developed using murine hybridoma technology. More recently other species such as rabbits have been developed to produce monoclonal antibodies. In order to avoid exposing the patient to foreign proteins (e.g. murine antibody) the monoclonal antibodies are often “humanized” by replacing certain portions of the antibody protein with human material. The monoclonal antibodies can be purified using standard down-stream processing techniques such as affinity binding to Protein A. These and other methods of developing and purifying monoclonal antibodies are known to those skilled in the art and are considered to be within the scope of this invention

Recombinant antibodies are produced using genetic engineering technology. Typically a wide variety of antibody binding domains are expressed as membrane surface or viral coat proteins (phage display). The antibody binding domain that binds to the desired target (antigen) is then isolated along with the corresponding genes which can be sequenced and introduced into various expression hosts (e.g. bacterial, yeast, insect, or mammalian cells) and used to produce a high yield of antibodies. The recombinant antibodies can be purified using standard down-stream processing techniques such as His-tagging the antibodies and isolating them using immobilized metal affinity chromatography. These and other methods of developing and purifying recombinant antibodies are known to those skilled in the art and are considered to be within the scope of this invention.

### Aptamer as Binding Agent

An aptamer is a single stranded DNA or single stranded RNA molecule that is synthesized to specifically bind to its target. Aptamers are small (i.e. 40 to 100 bases), synthetic oligonucleotides. They may be composed as a single-stranded DNA chain (ssDNA) or a single-stranded RNA chain (ssRNA). Each aptamer has a unique configuration as a result of the composition of the nucleotide bases in the chain causing the molecule to fold in a particular manner. Because of their folded structure each aptamer will bind selectively to a particular epitope in a manner analogous to an antibody binding to its antigen. In order to improve stability against nucleases found in vivo the oligonucleotides comprising the aptamer may be modified to avoid nuclease attack. They may for example be synthesized as L-nucleotides instead of D-nucleotides and thus avoid degradation from nucleases present in blood.

Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand. Once the nucleotide sequence is identified increased quantities of that aptamer can be synthesized. Since the SELEX was first introduced a variety of other methods and variations of producing aptamers have been developed. These methods are known to those of skill in the art and are considered to be within the scope of this invention.

### Binding Peptide as Binding Agent

A binding peptide is comprised of a chain of aminoacids that are synthesized and selected to target the harmful factor. There are various methods for preparing synthetic or biological peptide libraries composed of up to a billion different sequences, and for identifying a particular peptide sequence that will target a particular epitope. Typically a large number of different peptide sequences are allowed to react with the target and the peptide with the highest binding affinity is isolated and sequenced. Once the binding peptide sequence is identified increased quantities of that binding peptide can be produced by synthesis or using genetic engineering technology. The means of producing synthetic or biologically derived peptides are known to those of skill in the art and are considered to be within the scope of this invention.

### Support Matrix.

The binding agents in this invention are immobilized by covalently attaching them to an insoluble support matrix. Typically the support matrix is in the form of micron sized beads. Most often these are cross-linked agarose beads, but other types of beads such as acrylic beads and polystyrene beads can also be used. The essential requirements for the support matrix is that it must be insoluble, biocompatible, and have a large surface area to which the binding agent can be attached. The different binding agents are covalently attached to different batches of beads using coupling methods that are well-known to those of skill in the art. The different batches of binding agent coated beads are then mixed together before being placed in the apheresis cartridge. The beads are contained within the targeted apheresis cartridge by a retaining microporous top membrane, and a retaining microporous bottom membrane. The pore size of the retaining membranes are large enough to permit through passage of blood or plasma but smaller than the coated beads which are therefore retained within the device. After the proinflammatory factors are bound out, the treated blood or plasma is returned to the patient.

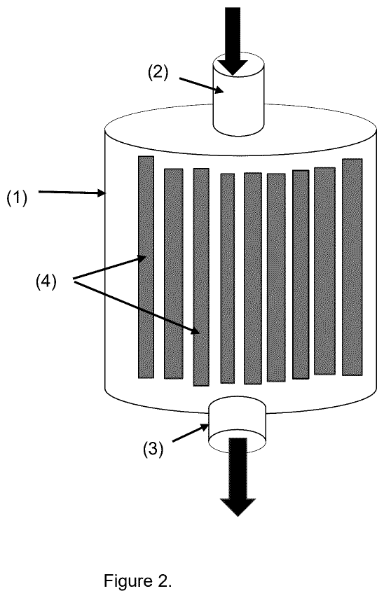

In one embodiment of this invention the support matrix is a microporous membrane. The support membrane will have a large surface area to which the binding agents can be attached. It may be folded or pleated in order to fit within the targeted apheresis device. The apheresis device in FIG. 2 comprises a cartridge (1) that has an inlet port (2) for blood or plasma to enter, and an outlet port (3) for blood or plasma to exit and be returned to the patient. In this invention the support membrane is preferably in the form of microtubules (4) such as that used in therapeutic apheresis devices. However, in contrast to therapeutic apheresis where the membrane is used to separate plasma from whole blood the membrane in this invention it is not used as a filter membrane, but instead it is used solely as a support matrix that has a large surface area to which the binding agents can be covalently attached. For example, the membrane surface area reported by manufactures of membranes used in therapeutic apheresis range from 0.2 m² to 2.0 m². However, it is important to note that the surface area of the support membrane upon which the binding agents are immobilized is actually much larger because of the presence of numerous pores in the membrane. The internal surface area of these pores provides for a very large surface area for attachment of the binding agent in addition to the outer surface area of the membrane. There are a variety of apheresis membranes available that can be adapted to facilitate covalent coupling of the binding agents to their surfaces. For example, they may be made from: cellulose diacetate (Plasma-APOS); polypropylene (Fenwal CPS-10); polysulfone (Sulflux-FS); polymethylmethacrylate (Plasmax-PS05) or combinations of these compounds. There are a variety of means whereby their surfaces can be functionalized to permit the covalent attachment of the binding agent to the surface.

### Immobilized Binding Agents.

There are a variety of methods for covalently attaching the binding agent to the support matrix. On surfaces that are aminated or carboxylated, covalent coupling is achieved using bifunctional cross-linkers that couple the amine or carboxyl group on the surface to a functional group, such as an amine or sulfhydryl, on the biomolecule. Selection of the cross-linker will determine the type of covalent bond that will be formed. Functional and covalently reactive groups, such as N-oxysuccinimide, maleimide and hydrazide groups, can also be grafted onto a suitable surface support via a photo-linkable spacer arm resulting in a reactive surface to covalently attach the binding agent. After the binding agent is attached to the support matrix any remaining active surfaces can be blocked using a solution of human albumin or similar blocking material. Another method for covalent attachment is to treat the support matrix with 3-aminopropyltriethoxysilane (APTES) and to cross-link the binding agent to the APTES functionalized surface using 1-Ethyl-3-(3-dimethylaminopropyl) carbodiimide (EDC). After the binding agent is attached to the support matrix any remaining active surfaces can be blocked using a solution of human albumin or similar blocking material. There are numerous established methods of attaching antibodies, binding peptides and aptamers to a variety of different surfaces. The method selected will depend on the moiety to be attached and the physical and chemical composition of the support matrix. These and other means of attachment are known to those of skill in the art and are considered to be within the spirit and scope of this invention.

### Targeted Apheresis

The Targeted Apheresis device in this invention comes in several configurations depending on whether the binding agents are coated on beads or on a support membrane; and also depending on whether whole blood or plasma is used. Note however that all versions of the targeted apheresis device utilize the same immobilized binding agents. The main differences between them are whether the support matrix are beads or a membrane; and if beads are used then the size of the beads and the smaller pores of the membranes used to retain them within the device. The following examples are presented to illustrate the underlying rationale for each version of the targeted apheresis device.

Example 1. In one embodiment of this invention when plasma is used the plasma fraction is first separated from the cellular elements in whole blood using differential centrifugation or membrane filtration. The procedures for preparing plasma using differential centrifugation or membrane filtration are known to those of skill in the art. The targeted apheresis device will typically contain a mixture of different binding agent coated beads that are about 30 microns in diameter. The beads are retained within the device by a top and bottom microporous membrane with pores that are smaller than the beads (e.g. pores are <10 microns in diameter). This configuration will allow plasma to flow through the device and contact the binding agent coated beads which will bind out proinflammatory factors before the treated plasma is returned to the patient. The binding agent coated beads being larger than the pores of the membranes will be retained within the device. Those of skill in the art will know that different sizes of beads and microporous membranes may be used, provided that the pores in the retaining membranes are smaller than the binding agent coated beads. (FIG. 1)

Example 2. In one embodiment of this invention when whole blood is used, the pores of the retaining membranes must be significantly larger than the size of red and white blood cells; but smaller than the size of the binding agent coated beads. For example, the pores in the retaining membranes could be 50-100 microns in diameter; while the binding agent coated beads could be 200-300 microns in diameter. The large beads in this embodiment of this invention are cross-linked agarose beads that are permeated with large pores that will allow entry of large molecules such as antibodies or aptamers or binding agents; and therefore these molecules when employed as binding agents will also be covalently attached to the internal pores of the beads. Therefore when the patient's blood comes into contact with these beads the CRP, C1q and TNF present will bind to both the external and internal binding agent coated surfaces of the beads. This configuration will allow whole blood to flow through the device and contact the binding agent coated beads which will bind out proinflammatory factors before the treated blood is returned to the patient. The binding agent coated beads being larger than the pores of the retaining membranes will be retained within the device. Those of skill in the art will know that different sizes of beads and matching retaining membranes may be used, as long as the membrane pores are significantly larger than the size of red and white blood cells; but smaller than the size of the binding agent coated beads. (FIG. 1).

Example 3. In one embodiment of this invention when the binding agents are attached to an insoluble supporting matrix that is a membrane, both plasma and whole blood can be processed through said targeted apheresis device without the need of retaining membranes. Instead there is unrestricted flow of plasma or blood through the apheresis device. (FIG. 2).

### Composition of the Targeted Apheresis Device.

For purpose of illustration only, and not as a limitation, this invention describes the composition and process for preparing a typical Targeted Apheresis Device. Those of skill in the art will be cognizant from the disclosures in this invention that there are many changes and modifications that can be made to the composition of said device without departing from the spirit and scope of this invention. Said changes are therefore considered to lie within the scope of this invention.

A typical Targeted Apheresis Device will consist of a rigid biocompatible cartridge containing a mixture of different batches of binding agent coated beads. A first batch of beads are coated with an anti-CRP binding agent; a second batch of beads are coated with an anti-C1q binding agent; and a third batch of beads are coated with an anti-TNF binding agent. The beads are typically cross-linked agarose beads. In this example the binding agents used are a) an anti-CRP antibody; b) an anti-C1q antibody; and c) an anti-TNF antibody.

There are a large variety of monoclonal and polyclonal anti-CRP antibodies that are commercially available. In this example the anti-CRP antibody is a monoclonal mouse anti-CRP antibody that binds to human CRP. The antibody is covalently attached to agarose beads using a commercially available 1 ml affinity column containing NHS-activated agarose beads (HiTrap Affinity Column, GE Healthcare Life Sciences). First, 1 mg of the antibody is dissolved in 1 ml of coupling buffer (0.2 M NaHCO₃, 0.5 M NaCl, pH 8.3). Next 6 ml of ice-cold 1 mM HCl is flowed through the column to replace the isopropanol in which the beads were packed. Then immediately 1 ml of the antibody dissolved in coupling buffer in slowly injected to the top of the column and the coupling is allowed to proceed at room temperature for 30 minutes. The column is then alternately washed with 6 ml of buffer A (0.5 M ethanolamine, 0.5 M NaCl pH 8.3) and 6 ml of buffer B (0.1 M sodium acetate, 0.5 M NaCl, pH 4). Finally, the column is washed with 6 ml of neutral buffer (0.05 M NaH₂PO₄, 0.1% NaN₃ pH 7); and stored at 4 C.

There are a large variety of monoclonal and polyclonal anti-C1q antibodies that are commercially available. In this example the anti-C1q antibody is a monoclonal antibody that binds to human C1q. The anti-C1q antibody is covalently attached to a second affinity column of NHS-activated agarose beads using the same procedure as that described earlier for the anti-CRP antibody.

There are a large variety of monoclonal and polyclonal anti-TNF antibodies that are commercially available. In this example the anti-TNF antibody is a monoclonal antibody that binds to human TNF. The anti-TNF antibody is covalently attached to a third affinity column of NHS-activated agarose beads using the same procedure as that described earlier for the anti-CRP antibody.

The binding capacity of each of the columns to their specific harmful factor i.e. CRP, or C1q, or TNF, is measured by preparing a sample containing a known quantity of that harmful factor in a physiological buffer and slowly injecting 1 ml of that spiked sample into its respective column. Then 2 ml of the plain buffer is injected into the column and the filtrate collected. The amount of the harmful factor in the 2 ml filtrate is measured using a standard enzyme-linked immunosorbent assay (ELISA) and subtracted from the known amount of harmful factor in 1 ml of the spiked sample to give a value of the amount that was bound out by the column.

Another method of measuring the amount of harmful factor bound is to wash out any unbound material by flowing 2 ml of plain buffer through the column. Then 2 ml of an elution buffer (i.e. 0.1 M glycine-HCl, pH 2.5) is flowed through the column and the eluate collected. The amount of harmful factor present in the eluate is measured using a standard ELISA for that particular harmful factor. This is a direct measurement of the harmful factor that was bound out by the targeted apheresis device.

The targeted apheresis device of this invention comprising a plurality of different immobilized binding agents is prepared by mixing the anti-CRP antibody coated beads, and the anti-C1q antibody coated beads, and the anti-TNF antibody coated beads together within the targeted apheresis device. The amount of each batch of binding agent beads to be mixed will vary depending upon the amount of each harmful factor that needs to be removed from the patient's blood; and the binding efficiency of the binding agent coated beads for that particular harmful factor. In practical terms to ensure optimum binding efficiency an excess of binding agent in relation to its target ligand is used in the apheresis device; and different ratios of the different batches of binding agent coated beads will be tested for binding efficiency in order to arrive at a final determination of the amounts of each batch of coated beads to be used in the finalized targeted apheresis device.

Example 4. In one embodiment of this invention a dual targeted apheresis cartridge system is used, wherein two apheresis cartridges are arranged in parallel and used alternatively such that one cartridge is regenerated while the other is in use. The cartridge to be regenerated is treated with elution buffer (e.g. glycine/HCl pH 2.5) to elute off the bound harmful factors and then washed with neutralizing buffer before it is reused. The dual cartridge system is particularly useful when large quantities of harmful factors need to be removed. There are a number of commercially available therapeutic apheresis machines that use this type of dual apheresis cartridge system (e.g. Liposorber®, Kaneka Medical Products America).

It will be obvious to those of skill in the art that the procedure for developing a Targeted Apheresis Device wherein the binding agents are antibodies can be modified by replacing said antibodies with their functionally equivalent aptamers or binding peptides. Said changes are therefore considered to lie within the spirit and scope of this invention.

This invention further teaches that there are in addition to multiple proinflammatory factors circulating in the blood; that there are also a variety of proinflammatory leukocytes in the blood that will migrate to the tissue injury site and participate in the inflammatory process. These include lymphocytes, granulocytes, macrophages, mononuclear cells, neutrophils, eosinophils, and mast cells. Removal of these proinflammatory cells using apheresis could further ameliorate disease symptoms and remission of the disease.

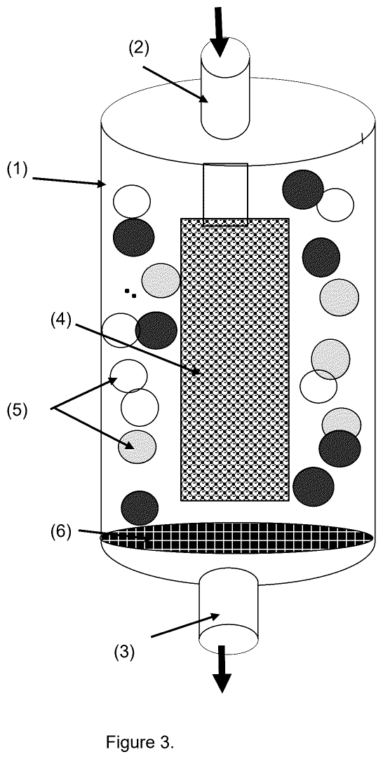

Example 5. In one embodiment of this invention there is a single targeted apheresis cartridge that is designed to simultaneously remove both the leukocytes and proinflammatory factors from the patient's blood. (FIG. 3). It consists of a single cartridge (1) with an inlet port (2) to allow blood to enter, and an outlet port (3) to allow blood to exit. The device comprises a biocompatible in-depth filter mesh (4) with pores than will allow red blood cells to filter through while retaining leukocytes that will bind to or are trapped within the mesh. The in-depth filter has a gradient of decreasing pore sizes from about 50 microns down to about 10 microns. After filtering through the leukocyte depletion filter the filtered blood then comes into contact with a mixture of large (e.g. 150-300 microns) binding agent coated beads (5) that will bind out CRP, C1q and TNF. The large beads in this embodiment of this invention are cross-linked agarose beads that are permeated with large pores that will allow entry of large molecules such as antibodies or aptamers or binding agents; and therefore these molecules when employed as binding agents will also be covalently attached to the internal pores of the beads. Therefore when the patient's blood comes into contact with these beads the CRP, C1q and TNF present will bind to both the external and internal binding agent coated surfaces of the bead. In this example, the anti-CRP binding agent coated beads are shown in white; the anti-C1q binding agent coated beads are shown in black; and the anti-TNF binding agent coated beads are shown in grey. There is a retaining microporous membrane (6) with large pores (e.g. 50-100 microns) at the bottom of the device that will allow the treated blood to flow out of the device while retaining the beads within the device. The treated blood is returned to the patient. Removing the proinflammatory leukocytes in addition to removing a plurality of harmful proinflammatory factors from the patient's blood will inhibit pathogenic inflammation and prevent exacerbation of the disease.

### Discussion

There is a general rule of thumb that when blood is processed using apheresis that for every 1.5 body volume of blood that is processed about 50 percent of the harmful factors are bound out; and if 3.0 body volumes of blood is processed then about 70 percent of the harmful factors are bound out. Further processing of more than three body volumes of blood is generally not done as it results in only incremental removal of harmful factors.

In order to suppress inflammation especially during the acute phase of a disease or following tissue injury, the patient's blood is tested repeatedly according to an accelerated schedule (e.g. daily) in order to measure the level of one or more harmful factors to determine if it reaches a level that will require additional treatment using targeted apheresis.

This invention teaches that the beneficial therapeutic effect obtained by removal of inflammatory factors using targeted apheresis could be further enhanced and extended by administering anti-inflammatory medications following apheresis. For example, medications such as corticosteroids have shown to be of benefit in treating inflammatory bowel disease. Also biologics such as anti-TNF antibody have been used to treat inflammation in rheumatoid arthritis and also to treat inflammatory bowel disease. These types of anti-inflammatory medications when used in conjunction with targeted apheresis could significantly extend the duration of disease remission.

This invention teaches that targeted apheresis to remove multiple proinflammatory factors would have a therapeutic effect upon a variety of inflammatory disease; and/or for diseases that had a harmful inflammatory component and/or following tissue injury such as heart attack or stroke. In one embodiment of this invention the removal of proinflammatory leukocytes combined with the removal of multiple proinflammatory factors will have an enhanced therapeutic benefit in treating a variety of inflammatory diseases and/or diseases that have a harmful inflammatory component. Further, that the beneficial therapeutic effect of said apheresis treatment described in this invention could be enhanced when combined with anti-inflammatory medications such as corticosteroids, or biologics. Targeted apheresis treatment combined with anti-inflammatory medication may result in long-term remission of many diseases where there is pathogenic inflammation involved in the disease process.

Those of skill in the art would be cognizant from the disclosures in this invention that there are various modifications and changes that can be made in the development of targeted apheresis devices similar to those examples described herein without departing from the spirit and scope of this invention. Said modifications and changes are therefore considered to lie within the scope of this invention.

What is claimed is:

1. A targeted apheresis device for removing a plurality of proinflammatory factors from a patient's blood or plasma; wherein said device is composed of a cartridge containing multiple immobilized binding agents that will specifically bind out a) C-Reactive Protein (CRP); and b) C1q component of complement; and c) Tumor Necrosis Factor (TNF); and optionally d) one or more other complement component, and/or one or more other proinflammatory cytokine.

2. A targeted apheresis device for removing a plurality of proinflammatory factors from a patient's blood or plasma according to claim 1, wherein the binding agent is an antibody, or an aptamer, or a binding peptide.

3. A targeted apheresis device for removing a plurality of proinflammatory factors from a patient's blood or plasma according to claim 1, wherein the support matrix is microbeads, or a microporous membrane preferably of the microtubular type used in therapeutic apheresis.

4. A targeted apheresis device for removing a plurality of proinflammatory factors from a patient's blood or plasma according to claim 1, wherein the patient in need will receive one or more targeted apheresis treatments.

5. A targeted apheresis device for removing a plurality of proinflammatory factors from a patient's blood; wherein said device is composed of a cartridge containing: a) a leukocyte reduction filter; and b) a plurality of immobilized binding agents that will bind out: C-Reactive Protein (CRP); and C1q component of complement; and Tumor Necrosis Factor (TNF); and optionally c) one or more other complement component, and/or one or more other proinflammatory cytokine.

6. A targeted apheresis device for removing a plurality of proinflammatory factors from a patient's blood according to claim 5, wherein the patient in need will receive one or more targeted apheresis treatments.
